(19) United States
(12) Patent Application Publication
(10) Pub. No.: US 2006/0111363 A1
(43) Pub. Date: May 25, 2006

(54) Treatment of uveal melanoma

(76) Inventors: Charlotta All-Ericsson, Bromma (SE); Olle Larsson, Taby (SE)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/531,967

(22) PCT Filed: Oct. 20, 2003
(86) PCT No.: PCT/EP03/11601

§ 371 Date: Sep. 13, 2005

(30) Foreign Application Priority Data
Oct. 21, 2002 (GB) 0224455.6

(51) Int. Cl. A61K 31/506

(52) U.S. Cl. 514/252.18

(57) ABSTRACT

The present invention pertains to the use of the Compound I of the following formula 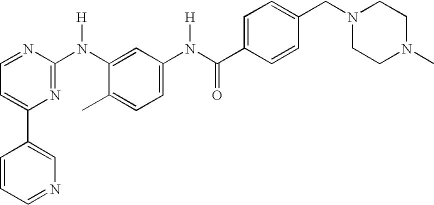 or a pharmaceutically acceptable salt thereof for the manufacture of a medicament for the treatment of uveal melanoma.

The invention relates to the use of 4-(4-methylpiperazin-1-ylmethyl)-N-[4-methyl-3-(4-pyridin-3-yl)pyrimidin-2-ylamino)phenyl]-benzamide (hereinafter referred to as “Compound I”) or a pharmaceutically acceptable salt thereof for the manufacture of pharmaceutical compositions for the treatment of uveal melanoma, to the use of Compound I or a pharmaceutically acceptable salt thereof in the treatment of uveal melanoma, to a method of treating warm-blooded animals including mammals, especially humans, suffering from uveal melanoma by administering to a said animal in need of such treatment a dose effective against said disease of Compound I or a pharmaceutically acceptable salt thereof.

Uveal melanoma is the most common primary intraocular tumor in adults with an annual incidence of 6 cases per million. Uveal melanoma metastasizes preferentially to the liver, and the prognosis for affected patients is extremely poor. The average survival time in uveal melanoma is only 2-5 months after detected liver metastases. In contrast to improved survival rates in a variety of cancers where there is a continuing evolution toward early detection and management, the survival rate with uveal melanoma has changed little during the past few decades (Diener-West et al., 1992, Arch. Ophthalmol. 110:245-50). Uveal melanoma is usually treated by enucleation or irradiation both leading to a poor prognosis of patients. Moreover, radiation therapy induces numerous complications such as neovascular glaucoma. Chemotherapy and/or immunotherapy has been used in the treatment of metastatic melanoma but the results have generally been disappointing, with a median survival time after treatment of 5 to 8 months (Pyrhonen S. 1998, Eur. J. Cancer 34, Suppl. 3:S27-30). There is a need for efficient and less traumatic treatments of uveal melanoma.

Compound I is 4-(4-methylpiperazin-1-ylmethyl)-N-[4-methyl-3-(4pyridin-3-yl)pyrimidin-2 -ylamino)phenyl]-benzamide having the following formula

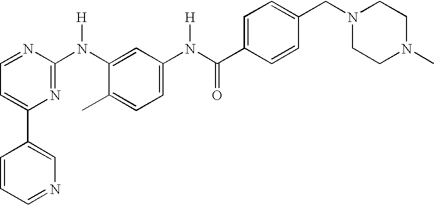

Compound I free base, its acceptable salts thereof and its preparation are disclosed in the European granted patent 0564409, hereby incorporated by reference. Compound I free base corresponds to the active moiety.

The monomethanesulfonic acid addition salt of Compound I (hereinafter referred to as “Salt I”) and a preferred crystal form thereof (the beta crystal form) are described in PCT patent application WO99/03854 published on Jan. 28, 1999, hereby incorporated by reference.

Compound I is an inhibitor of platelet-derived growth factor receptors alpha and beta (PDGFRs α and β), Bcr—Abl and c-kit tyrosine phosphorylation. It has been shown that c-kit is expressed in epidermal melanocytes but in primary melanomas loss of the receptor is observed in more invasive lesions (Natali et al., Int. J. Cancer. 1992, 52:197-201). C-kit is expressed in normal melanocytes but both the protein and the RNA expression is lost in most primary and metastatic melanoma cell lines (Lassam and Bickford, 1992, Oncogene, 7:51-6).

Since c-kit is down-regulated in melanomas of cutaneous origin, it was unexpectedly found that 84 specimens among the 134 specimens of uveal melanomas expressed c-kit and that treatment of uveal melanoma cell lines with Compound I or a pharmaceutically acceptable salt thereof blocks c-kit auto-phosphorylation and results in cell death.

Surprisingly, it was found that Compound I is particularly useful for the treatment of uveal melanoma. Compound I promotes in vitro cell death on four uveal melanoma cell lines, possibly through the inhibition of c-kit phosphorylation and/or PDGFR.

The term “uveal melanoma” means any tumor deriving from the uvea. The uvea consists of the iris (the colored or pigmented part surrounds the pupil, the opening that controls the amount of light that enters the eyeball), the choroid (a thin, pigmented layer lining the eyeball that nourishes the retina and the front of the eye with blood), the ciliary body (contains the muscles inside the eye that change the shape of the lens so that the eye can focus on near or distant objects and cells that produce aqueous humor, fluid in the eye).

The invention thus relates to the use of a c-kit inhibitor or a pharmaceutically acceptable salt thereof as a drug against uveal melanoma. Most preferably, the invention relates in the use of Compound I or a pharmaceutically acceptable salt thereof as a drug against uveal melanoma.

The present invention further pertains to the use of Compound I or a pharmaceutically acceptable salt thereof for the manufacture of a medicament for the treatment of uveal melanoma.

The present invention also relates to the use of Compound I or a pharmaceutically acceptable salt thereof for the manufacture of a medicament for the treatment of metastasing uveal melanoma.

The pharmaceutical compositions according to the present invention can be prepared in a manner known per se and are those suitable for enteral, such as oral or rectal, and parenteral administration to warm-blooded animals, including man, comprising a therapeutically effective amount of at least one pharmacologically active ingredient, alone or in combination with one or more pharmaceutically acceptable carries, especially suitable for enteral or parenteral application. The preferred route of administration of the dosage forms of the present invention is orally.

The invention relates to a method of treating a warm-blooded animal having uveal melanoma comprising administering to said animal in need for such a treatment Compound I or a pharmaceutically acceptable salt thereof, in a quantity which is therapeutically effective against uveal melanoma.

The invention relates to a method for administering to a human subject suffering from uveal melanoma an acid addition salt of 4(4-methylpiperazin-1-ylmethyl)-N-[4methyl-3-(4-pyridin-3-yl)pyrimidin-2-ylamino)phenyl]-benzamide and preferably Salt I, the monomethanesulfonate salt of Compound I.

In one embodiment of the invention, the monomethane sulfonate salt of Compound I is in the beta crystal form.

The person skilled in the pertinent art is fully enabled to select relevant test models to prove the beneficial effects mentioned herein on uveal melanoma. The pharmacological activity of such a compound may, for example, be demonstrated by means of the Examples described below, by in vitro tests and in vivo tests or in suitable clinical studies. Suitable clinical studies are, for example, open label non-randomized, dose escalation studies in patients with metastatic uveal melanoma. The efficacy of the treatment is determined in these studies, e.g., by evaluation of the tumor sizes every 4 weeks, with the control achieved on placebo.

The effective dosage of Compound I may vary depending on the particular compound or pharmaceutical composition employed, on the mode of administration, the type of the uveal melanoma being treated or its severity, e.g. metastasing uveal melanoma. The dosage regimen is selected in accordance with a variety of further factors including the renal and hepatic function of the patient. A physician, clinician or veterinarian of ordinary skill can readily determine and prescribe the effective amount of compounds required to prevent, counter or arrest the progress of the condition.

Depending on age, individual condition, mode of administration, and the clinical picture in question, effective doses, for example daily doses of Compound I or a pharmaceutically acceptable salt thereof corresponding to 100 to 1000 mg of the free base as active moiety, especially 800 mg, are administered to warm-blooded animals of about 70 kg body weight. Preferably, the warm-blooded animal is a human. For patients with an inadequate response to daily doses, dose escalation can be safely considered and patients may be treated as long as they benefit from treatment and in the absence of limiting toxicities.

The invention relates also to a method for administering to a human subject suffering from uveal melanoma, Compound I or a pharmaceutically acceptable salt thereof, which comprises administering a pharmaceutically effective amount of Compound I or a pharmaceutically acceptable salt thereof to the human subject once daily for a period exceeding 3 months. The invention relates especially to such method wherein a daily dose of 100 to 1000 mg, e.g. 400 to 800 mg, e.g. 400 mg, 600mg, 800 mg, preferably 800 mg, of Compound I is administered to an adult.

### EXAMPLE 1

### Dose Response for the Treatment of 4 Uveal Melanoma Cell Lines with Salt I after a 48 Hours Exposure

Salt I anti-proliferative effects are determined by in vitro culture of four uveal melanoma cell lines in the presence of Salt I.

Cell culture: Four cell lines obtained from human primary uveal melanomas (OCM-1, OCM-3, 92-1 and mel 202) are used. They are kindly provided by Dr Martine Jager (Leiden University Medical Center, Leiden, The Netherlands).

Cell proliferation kit II is purchased from Roche Diagnostic GmbH (Mannheim, Germany). The test is based on a colorimetric change of the yellow tetrazolium slat XTT into orange formazan dye by the respiratory chain of viable cells (Roehm et al., 1991, J. Immunol. Assay, 142:257-265).

Results:

A: Dose Response of Salt I on 9-21, OCM-1, mel 202 and OCM-3 Cells

| [Salt I] in μM | | | 0 | 0.15 | 0.3 | 0.6 | 1.25 | 2.5 | 5 | 10 |
|---|---|---|---|---|---|---|---|---|---|---|
| UM 92-1 | 1 | 24 h | 100 | 97 | 98 | 95 | 97 | 93 | 83 | 70 |
| | | 48 h | 100 | 87 | 85 | 79 | 50 | 23 | 15 | 0 |
| | 2 | 24 h | 100 | 107 | 91 | 102 | 97 | 102 | 75 | 83 |
| | | 48 h | 100 | 92 | 90 | 71 | 46 | 18 | 22 | 5 |
| OCM-1 | 1 | 24 h | 100 | 90 | 95 | 87 | 70 | 78 | 63 | 50 |
| | | 48 h | 100 | 98 | 62 | 40 | 24 | 15 | 27 | 23 |
| | 2 | 24 h | 100 | 102 | 97 | 82 | 80 | 75 | 63 | 52 |
| | | 48 h | 100 | 73 | 59 | 37 | 34 | 32 | 23 | 29 |
| mel 202 | 1 | 24 h | 100 | 100 | 98 | 92 | 83 | 98 | 97 | 78 |
| | | 48 h | 100 | 24 | 23 | 19 | 15 | 13 | 23 | 21 |
| | 2 | 24 h | 100 | 98 | 105 | 104 | 95 | 98 | 101 | 87 |
| | | 48 h | 100 | 5 | 14 | 17 | 22 | 11 | 15 | 7 |
| OCM-3 | 1 | 24 h | 100 | 100 | 100 | 100 | 97 | 95 | 85 | 87 |
| | | 48 h | 100 | 51 | 43 | 34 | 35 | 32 | 25 | 23 |
| | 2 | 24 h | 100 | 110 | 97 | 105 | 102 | 87 | 83 | 98 |
| | | 48 h | 100 | 58 | 39 | 27 | 25 | 14 | 11 | 11 |

To examine the anti-proliferative effects of Salt I on the uveal melanoma cells lines, OCM-1, OCM-3, 92-1 and mel 202, cells are incubated with different concentrations of the drug for 48 h, after which the level of cell viability is assayed. The above table shows the dose response of OCM-3 and 92-1.The results are given as percentages of living cells in the samples with Salt I as compared to a 100% survival of the cells in the samples that are not treated with Salt I. There is a drastic cell loss even at low concentrations of Salt I.

B) IC₅₀ of Salt I on Survival of 4 Uveal Melanoma Cell Lines (OCM-1, OCM-3, UM 92-1, mel 202) and 2 Skin Melanoma Cell Lines (BE and DFB)

| melanoma | uveal melanoma cell line | | | | skin cell line | |
|---|---|---|---|---|---|---|
| cell line | OCM-1 | OCM-3 | UM 92-1 | mel 202 | BE | DFB |
| IC₅₀ μM | 0.4 | 0.15 | 1.25 | 0.07 | >>20 | >>20 |

In the above table, the IC₅₀ doses of Salt I for survival of all 4 uveal melanoma cell lines and of the two skin melanoma cell lines are compared. The IC₅₀ values are 0.07-1.25 μM in the uveal melanoma cells, but >>20 μM in BE and DFB. There is a significant difference in Salt I responsiveness between the cell types, skin melanoma cell lines being no respondent to Salt I.

### EXAMPLE 2

### Expression of C-Kit in Uveal Melanoma

The expression of c-kit is investigated in uveal melanoma samples retrieved from patients by immunohistochemistry and western blot analysis.

A) Expression of C-Kit in Uveal Melanoma

The expression of c-kit in uveal melanomas is investigated by immunohistochemistry on 134 paraffin-embedded surgical specimens of primary uveal melanoma.

Monoclonal antibody: A mouse monoclonal antibody directed to the human c-kit (CD117) was purchased from DAKO (CA, USA).

Immunohistochemistry: Immunostaining is performed using the standard ABC-technique (Vector, Elite Standard Kit. cat. PK-6100). The results of c-kit staining are reporter as negative when no staining is present, low when less than 10% of melanoma cells are immunopositive, medium when 10 to 50% are stained and high when more than 50% of the melanoma cells are stained.

Results: Samples are grouped according to staining as: no positive cells, <10% positive cells, 10-50% positive cells and >50% positive cell as analyzed using an anti-c-kit monoclonal antibody.

| | c-kit expression | | | | |
|---|---|---|---|---|---|
| | (% of cells positively stained | | | | |
| | with anti-c-kit MAB) | | | | |
| | no positive cell | <10% | 10%-50% | >50% | |
| number of cases | 50 | 18 | 18 | 48 | (total) 134 |

A distinct immunoreactivity confined to the plasma membrane/cytoplasm of tumor cells is found in 64% of the cases (84 of 134). Interestingly, in 48 of 134 (36%) the majority of tumor cells express c-kit.

B) C-kit Expression in Frozen Samples and Skin Samples of Skin Melanoma.

To confirm the results from immunohistochemistry, the expression of c-kit is studied on 8 fresh-frozen samples from primary uveal melanoma and 6 fresh frozen samples from skin melanoma by Western blotting using an antibody specific for c-kit.

Western blotting: Preparation of cell membranes is performed essentially as described elsewhere (Carlberg et al. 1996). After centrifugation the material is dissolved in sample buffer for SDS-PAGE. The proteins are transferred overnight onto nitrocellulose membranes. Incubation with the primary antibody is performed for 1 hour at room temperature, followed by three washes with PBS and incubation with a biotinylated secondary antibody (Amersham) for 1 hour. After a 15-min incubation with streptavidin-labeled horse peroxidase, detection is performed by enhanced chemiluminiscence. Signal is detected with Hyperfilm-ECL (Amersham).

Results: Six of the uveal melanoma specimens and none of the skin melanoma samples result in a positive signal (data not shown). This result suggests that Salt I might act through the inhibition of c-kit tyrosine phosphorylation to efficiently promote the cell death of uveal melanoma cells.

### EXAMPLE 3

### Capsules with 4-[(4-methyl-1-piperazin-1-ylmethyl)-N-[4-methyl-3 [[4-(3-pyridinyl)-2-pyrimidinyl]aminophenyl]benzamide methanesulfonate, Beta Crystal Form

Capsules containing 119.5 mg of Salt I corresponding to 100 mg of Compound I (free base) as active moiety are prepared in the following composition:

Composition:

| | |
|---|---|
| Salt I | 119.5 mg |
| Cellulose MK GR | 92 mg |
| Crospovidone XL | 15 mg |
| Aerosil 200 | 2 mg |
| Magnesium stearate | 1.5 mg |
| | 230 mg |

The capsules are prepared by mixing the components and filling the mixture into hard gelatin capsules, size 1.

### EXAMPLE 4

### Capsules with 4-(4-methyl-piperazin-1-ylmethyl)-N-[4methyl-3-[[4-(3-pyridindyl)-2-pyrmidinyl]amino]phenyl]benzamide methanesulfonate, Beta Crystal Form

Capsules containing 119.5 mg of Salt I corresponding to 100 mg of Compound I (free base) as active moiety are prepared in the following composition:

Composition:

| | |
|---|---|
| Salt I | 119.5 mg |
| Avicel | 200 mg |
| PVPPXL | 15 mg |
| Aerosil | 2 mg |
| Magnesium stearate | 1.5 mg |
| | 338.0 mg |

The capsules are prepared by mixing the components and filling the mixture into hard gelatin capsules, size 1.

### EXAMPLE 5

### Clinical Trials

This is an open label, phase II clinical trial testing SALT I in patients with uveal melanoma for which available evidence suggests an association with kit protein tyrosine kinase.

Patient/Disease population: Patients with uveal melanoma may be eligible to receive Salt I treatment if the following criteria are met relative to their disease:

the disease has proven to be refractory to standard therapeutic options or no conventional therapies of definitive benefit exist

tissue samples from the patient express CD117 (c-kit) as tested by immunohistochemical (IHC) assay.

Treatment: Patients with uveal melanoma receive Salt I initially at a dose corresponding to 800 mg per os/day (400 mg bis in diem) of Compound I. Dose levels may be escalated up to 500 mg bis in diem. (1000 mg per os/day) if no significant improvement in the disease occurs after the first eight weeks on therapy. Furthermore, changes in dosing may be based on progression of disease or on relevant laboratory evaluations. Any dose escalation may occur provided the patient may be benefiting from the increased dose of Salt I. Salt I therapy is be continued for as long as the patient is benefiting from treatment with Salt I, in the absence of any safety concerns.

Pharmacodynamic assessments: An overall objective assessment of disease will be performed according to the visit schedules. The 4-week assessment must include all identified disease-associated parameters, including scientific (radiology and laboratory results) and clinical evaluations.

FDG-PET scanning: Based on the putative mechanism of action of the drug, it is possible that a change in the metabolic profile of the tumor will be detectable before any tumor response (as measured by standard radiological methods) will occur. In order to investigate this possibility, a standard fluorodeoxyglucose (FDG) PET scan is an optional measure of this protocol to be implemented at baseline and after one month of treatment. Earlier FDG-PET scans, i.e. after 7 days of treatment, may also be recommended.

Dynamic MRI (magnetic resonance imaging): In order to investigate alterations of tumor vascular permeability and blood flow, a standard dynamic MRI procedure with contrast agent injection maybe performed.

### EXAMPLE 6

### Study

A study shows expression of PDGFRs in some uveal melanoma cell lines and samples. The IC₅₀ values for c-kit phosphorylation in uveal melanoma cells range from 0.8 to 2.5 μM. The IC₅₀ values for cell growth in uveal melanoma cells are ranging of about 0.15 to 1.25 μM. The little lower IC₅₀ values of Compound I for uveal melanoma cell survival compared to IC₅₀ values for effect on c-kit phosphoryation might be explained by inhibition of PDGFRs.

1. A method of treating a mammal suffering from uveal melanoma comprising administering to said mammal in need of such a treatment, a dose, effective against said disease a Compound I of the following formula 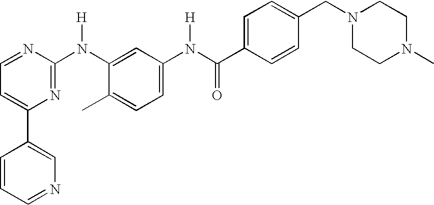
or a pharmaceutically acceptable salt thereof.

2. The method according to claim 1 wherein the uveal melanoma is a metatazing uveal melanoma.

3. The method according to claim 1 wherein the uveal melanoma expresses c-kit.

4. The method according to claim 1 wherein Compound I is in the form of the monomethanesulfonate salt.

5. The method according to claim 4 wherein the monomethanesulfonate salt of Compound I is in the beta crystal form.

6. The method according to claim 1 wherein Compound I is administered at a daily dose corresponding to 100 mg to 1000 mg of Compound I free base.

7. The method according to claim 6 wherein Compound I is administered once daily for a period exceeding 3 months.

8. (canceled)

9. Use of a c-kit inhibitor or a pharmaceutically acceptable salt thereof for the preparation of a medicament for the treatment of uveal melanoma.

10. Use according to claim 9 wherein the uveal melanoma is a metastazing uveal melanoma.
